(19) United States
(12) Patent Application Publication
(10) Pub. No.: US 2024/0307434 A1
(43) Pub. Date: Sep. 19, 2024

(54) USE OF LUTETIUM SALT COMPOUNDS FOR ANTIVIRAL EFFECT

(71) Applicants: Fikrettin Sahin, Istanbul (TR); Sevda Demir, Istanbul (TR)

(72) Inventors: Fikrettin Sahin, Istanbul (TR); Sevda DEMIR, Istanbul (TR)

(73) Assignees: Fikrettin Sahin, Istanbul (TR); Sevda Demir, Istanbul (TR)

(21) Appl. No.: 18/578,332

(22) PCT Filed: Jun. 13, 2022
(86) PCT No.: PCT/TR2022/050581

§ 371 Date: Jan. 11, 2024

(51) Int. Cl. A61K 33/244; A01N 59/16; A01P 1/00; A61P 31/14; A61P 31/20; A61P 31/22

(52) U.S. Cl. CPC A61K 33/244; A01N 59/16; A01P 1/00; A61P 31/14; A61P 31/20; A61P 31/22

(57) ABSTRACT

A lutetium salt compound for providing an antiviral effect against DNA and RNA viruses on infected cells, where the DNA and RNA viruses comprise Adenovirus, Poliovirus, Herpes Simplex Type 1, Herpes Simplex Type 2, Coronavirus, and Norovirus. Specifically, the lutetium salt compound includes lutetium borate, lutetium chloride, and lutetium nitrate.

### CROSS REFERENCE TO THE RELATED APPLICATIONS

This application is the national phase entry of International Application No. PCT/TR2022/050581, filed on Jun. 13, 2022, the entire contents of which are incorporated herein by reference.

### TECHNICAL FIELD

The present invention relates to salt compounds obtained from lutetium, such as lutetium borate, lutetium chloride and lutetium nitrate, which exhibit antiviral efficacy against Adenovirus, Poliovirus, Herpes Simplex Type 1 and Herpes Simplex Type 2, Coronavirus and Norovirus strains on infected suitable cell lines (HaCaT, Vero, Raw 264.7).

### BACKGROUND

Viruses are one of the major causes of diseases and deaths in the world and can infect all types of cellular life, including both eukaryotes and prokaryotes. Viruses consist of a protein structure called capsid on the outside and a genetic material in the form of single-or double-stranded DNA or RNA. Some viruses have also another structure, called the envelope, in addition to the capsid, which is composed of lipoproteins and has antigenic properties. Viruses, also defined as intracellular parasites, can use advanced host cells (bacteria, plant, or animal cells) to produce their viral proteins and genetic materials.

Various drugs have been developed to inactivate viruses or prevent them from reproducing. For an antiviral agent to be considered effective, it must directly affect the virus or its reproduction without affecting the host cell. The antiviral drugs widely used in the state of the art are examined under four main headings. These are drugs effective against influenza viruses, drugs effective against herpes viruses, anti-HIV drugs and immunomodulators used in antiviral therapy. Although these drugs are already in use, with number of serious side effects such as fatigue, vomiting, stomach upset, diarrhea, dizziness, toxic epidermal necrolysis, peripheral neuropathy, intraoral ulcers, esophageal ulcers, hepatomegaly, increasing diabetes, etc [1]. Furthermore, although precautions are taken with vaccines against some viral diseases that cause death, such as smallpox virus, there is no existing preventive or therapeutic method for persistent or emerging viruses. Therefore, there is a need for novel antiviral agents that can be used to overcome the side effects of existing antivirals, to administer combined treatment in patients who develop immunodeficiency or experience immunodeficiency, and in cases of antiviral resistance.

Lanthanides are composed of 15 elements called rare earth elements. In the 1960s, it was discovered that they exhibit pharmacological properties such as anticoagulation, anti-inflammatory, antibacterial, antiallergic and anticancer; and due to these properties, compounds containing Lanthanide (III) ions (rare earth metal ions) have been of interest since the 19th century [2]. Lanthanide (III) ion compounds play a very important role in medicine, especially in the diagnosis and treatment of cancer, and these compounds were also used as antibacterial agents for the treatment of tuberculosis in the early 20th century [3]. Lutetium is the last and smallest of the lanthanide family. Lutetium, which is a silvery metal as a pure element, is similar to calcium and magnesium in its reactivity. Lutetium and its compounds are involved in some applications in the petrochemical industry, such as making catalyst for cracking hydrocarbons or being used as an effective and recyclable catalyst for organic synthesis. In the field of healthcare, lutetium-177, which is the radioactive form of lutetium, is used to treat gastrointestinal tract cancers, including the stomach, pancreas and intestines. However, there is no application in the technical field for the use of lutetium for antiviral purposes.

European patent document with publication number EP2546839 B1, an application known in the known state of the art, discloses the carrier-free lutetium-177 compounds suitable for use in the medical field and the production method of the said compounds. The antiviral efficacy of lutetium is not disclosed in the document

Due to the aforementioned drawbacks and problems, there is a need for novel compounds that will exhibit antiviral effects in the current technical field.

### SUMMARY

The main objective of the present invention is to enable the use of lutetium salt compounds, whose antiviral effects on both DNA and RNA viruses have been detected within the scope of the invention, in broad-spectrum antiviral drug formulations. These compounds, whose antiviral property is detected, have the potential to be used for the treatment/prevention of human, animal, and plant diseases.

### BRIEF DESCRIPTION OF THE DRAWINGS

The “USE OF LUTETIUM SALT COMPOUNDS FOR ANTIVIRAL EFFECT”, which is realized to achieve the objective of the present invention, is illustrated in the accompanying figures, in which:

FIG. 1 is the illustration of the results of the cytotoxicity analysis with the concentration and cell viability variables. (PC: Positive Control, NC: Negative Control)

FIGS. 2A-2B are graphs of TCID₅₀ value of Herpes Simplex Type 1. (NC: Negative Control).

FIGS. 3A-3B are graphs of TCID₅₀ value of Herpes Simplex Type 2. (NC: Negative Control).

FIGS. 4A-4B are qRT-PCR results of Lutetium Borate against Herpes Simplex Type 1. (VC: Virus Control)

FIGS. 5A-5B are qRT-PCR results of Lutetium Borate against Herpes Simplex Type 2. (VC: Virus Control)

FIGS. 6A-6B are qRT-PCR results of Lutetium Chloride and Lutetium Nitrate against Herpes Simplex Virus Type 2. (VC: Virus Control)

FIGS. 7A-7I are microscope images showing the antiviral efficacy of the maximum non-toxic concentration of Lutetium Borate against HSV-1, HSV2, Adenovirus and Poliovirus for 72 hours. (Negative control: cell culture without virus, Virus controls: culture with only virus added on the cell, LBO₃ 50%: cell culture with substance applied on infected cells).

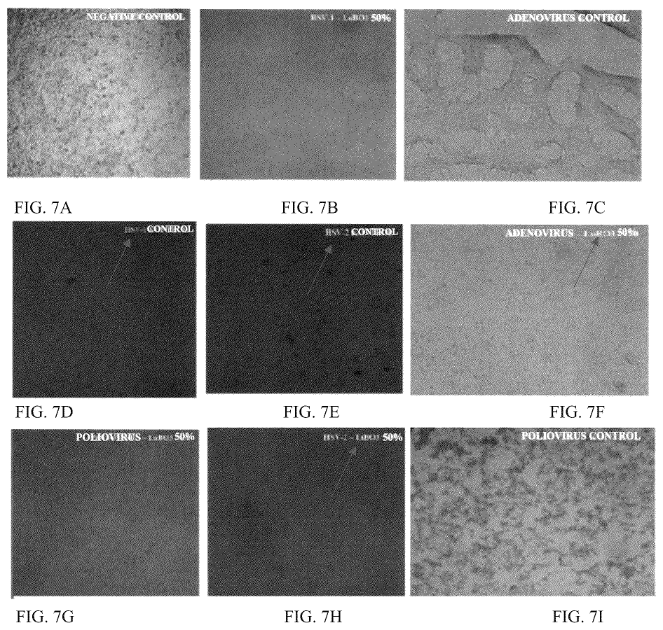

### DETAILED DESCRIPTION OF THE EMBODIMENTS

The present invention relates to the lutetium salt compounds, which exhibit antiviral efficacy against Adenovirus, Poliovirus, Herpes Simplex Type 1 and Herpes Simplex Type 2, Coronavirus and Norovirus strains on infected immortal homan keratinocyte cells (HaCaT). The lutetium salt compounds used in the treatment of the diseases mentioned in the invention are preferably lutetium borate, lutetium chloride, and lutetium nitrate. In the invention, other water-soluble and water-insoluble lutetium salt compounds formed with the lutetium element, which is used for providing antiviral effect against DNA and RNA virus strains, can be nitrate, sulfate, acetate, oxide, hydroxide, fluoride, carbonate, phosphate, oxalate, etc. Any one or combination of said Lutetium salt compounds can be used in any product and/or formulation produced for pharmaceutical, medical device, biocidal and cosmetic purposes (cream, spray, lotion, gel, capsule, tablet, toothpaste, mouthwash, mask, disinfectant, antiseptic, powder, polish, brightener, jelly, foam pomade).

In order to prove the efficacy of lutetium salts, the processes of the synthesis of lutetium salt compounds, cytotoxicity assay, determination of viral titration, antiviral efficacy analyses, quantitative real time PCR analysis and calculation of infectivity titer with TCID 50 were performed in the studies carried out within the scope of the invention. The said processes are explained in detail below.

Synthesis of lutetium borate: The lutetium Borate (LuBO3) produced in the laboratory environment was prepared to be at an amount of 750 ppm, using deionized water.

Lutetium nitrate solution: The lutetium nitrate hydrate ((Lu(NO₃)₃xH₂O, Cas #100641-16-5, Sigma) was prepared to be at an amount of 750 ppm, using deionized water.

Lutetium chloride solution: The lutetium chloride (LuCL_{3,} Cas #10099-66-8, Sigma) was prepared to be at an amount of 750 ppm, using deionized water.

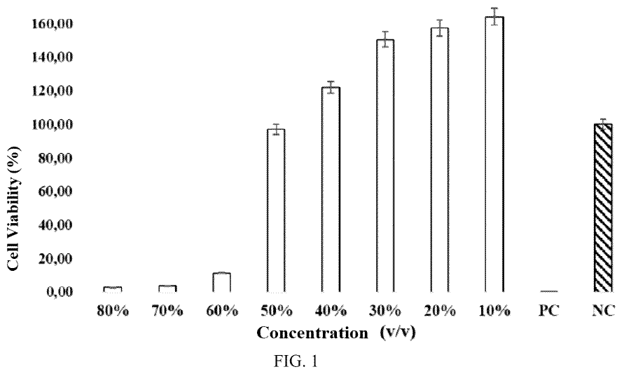

Cytotoxicity Assay: The effect of the prepared compounds on cell viability was measured by a substance called MTS based on mitochondrial dehydrogenase enzyme activity. In the said method, HaCaT (immortalized human keratinocyte cell) was prepared in medium and seeded in 96-well culture plates with 5000 cells per well. After the incubation period (24 hours), the medium on the cell was removed and the compounds at certain concentrations were diluted with medium and applied on the cell. The response of the cells to the toxicity of the molecules was detected by measuring cell viability after 72 hours. After the incubation period is completed, the MTS substance, which is added on the cells together with the medium, causes formation of colored formazan crystals as an indicator of cell viability. This color change was evaluated based on the absorbance measurement by using ELISA plate reader. The obtained results were analyzed. The results of the analysis are given in the graph in FIG. 1.

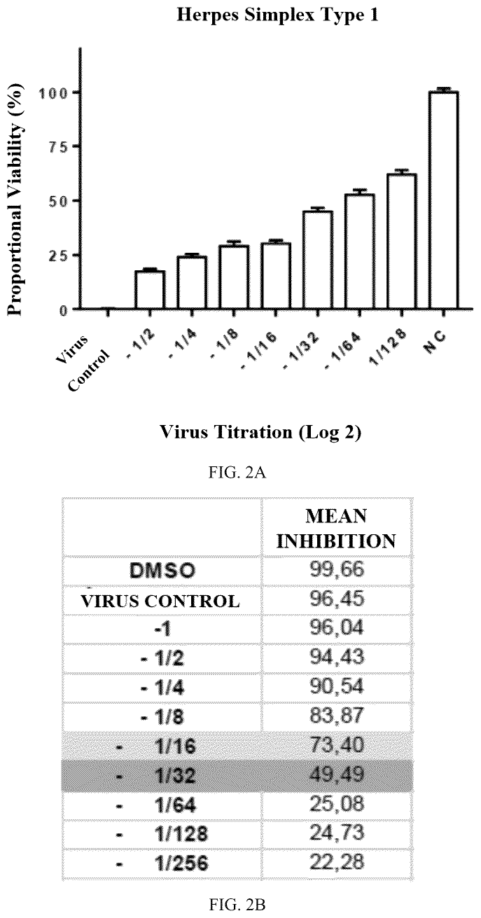

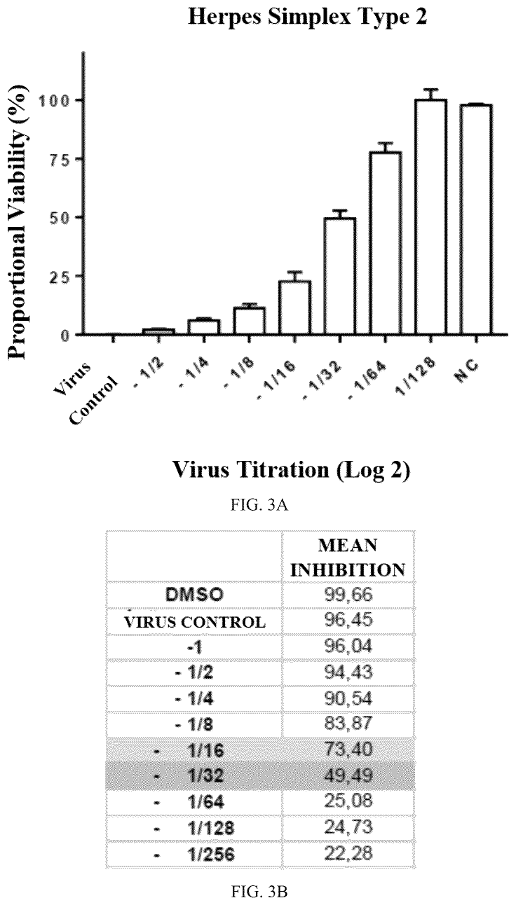

Determination of Viral Titration: Viral titration constitutes the most important step in any virologie research that requires the use of a specific amount of virus, and especially in the study for demonstrating the efficacy in potential antiviral agents. Colorimetric MTS method was used to determine viral titration because classical methods are quantitative and prone to subjective error. For this analysis, HaCat cells were seeded at 3×10⁴ cells and incubated at 37° C. for 24 hours. The next day, virus from the virus stock was prepared on ice on a logarithmic scale (Log 2) from 2-1 to 2-7. The medium on the monolayered cells was discarded and the cells were washed 3 times with PBS. 50 μl of the prepared virus dilutions were taken and added to each well in 6 replicates and incubated for two hours by stirring every twenty minutes to infect the cells. Then, the viruses on the cells were removed and the cells were washed three times with PBS. Virus media were added on the cells and incubated at 37° C. in CO₂ environment for 72 hours. DMSO (20%) for positive control and infected medium for negative control were aspirated, and 200 μl of virus medium containing 10% MTS was added to each well and incubated for 3 hours. The resulting color change was evaluated based on the absorbance measurement by ELISA plate reader at 490 nm (FIGS. 2A-2B and FIGS. 3A-3B).

Antiviral Efficacy Analyses: HaCaT cells were added to 48-well culture plates at 2×10⁵ cells per well and incubated at 37° C. for 24 hours. At this stage, the medium on the monolayered cells was discarded and the cells were washed 3 times with PBS. 100 μl of the viruses whose TCID50 value was determined were placed on the cells. The cells were incubated for two hours with stirring every twenty minutes to infect the cells. Then, the viruses on the cells were removed and the cells were washed three times with PBS. The concentrations of the substances whose non-toxic dose was determined were prepared with virus medium and added on the cell and incubated at 37° C. in CO₂ environment for 72 hours.

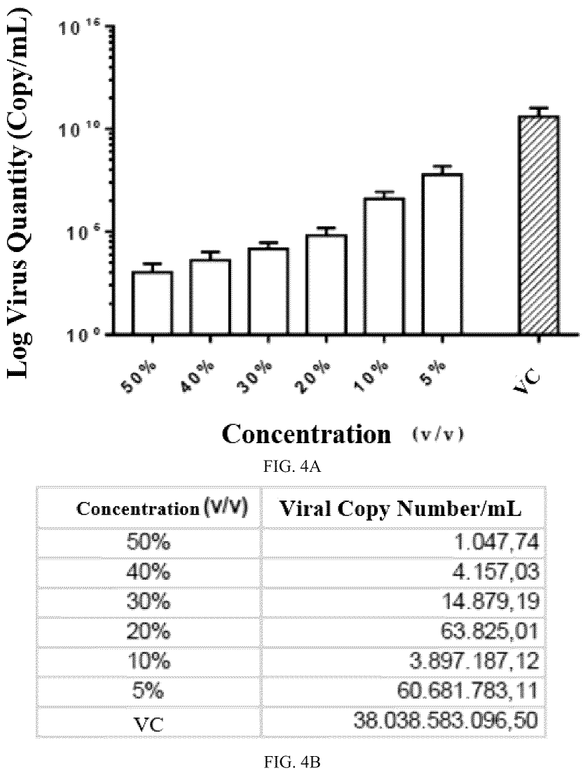

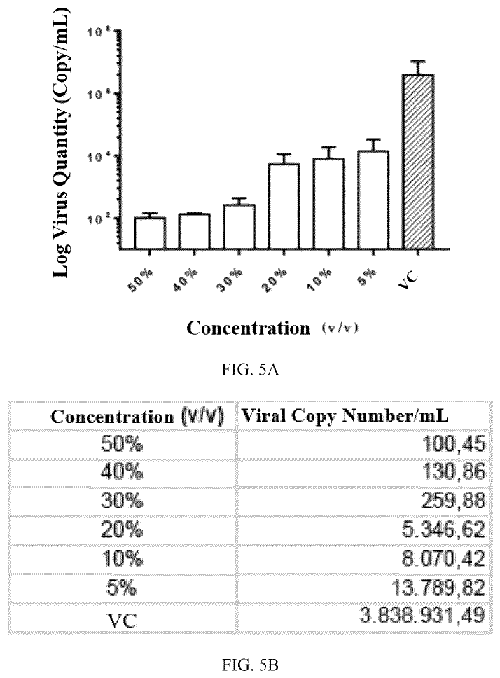

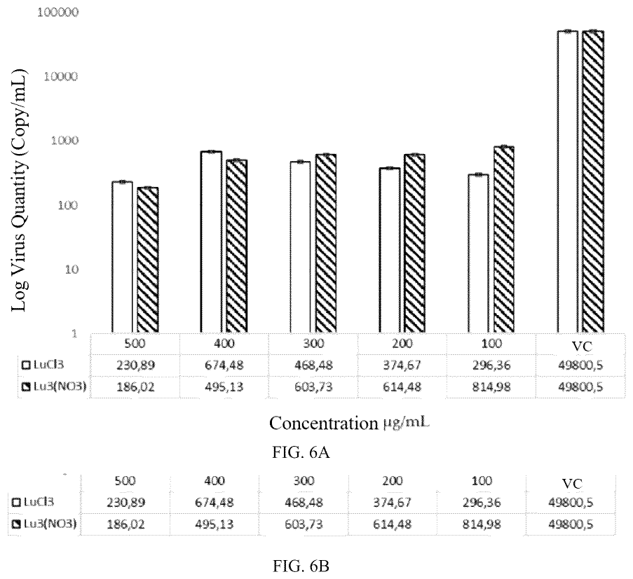

Quantitative Real Time PCR Analysis: The virus medium was taken into eppendorf tubes from the plates whose incubation is completed, centrifuged and the supernatant was transferred to new eppendorf tubes. Virus DNA was isolated from the collected supernatants according to the kit protocol (Viral nucleic acid kit, Roche). Then, PCR analysis was performed according to the quantitative PCR kit (HSV1, HSV2, Argene, Biomerieux) protocol (FIGS. 4A-4B, FIGS. 5A-5B, and FIGS. 6A-6B).

Calculation of Infectivity Titer with TCID 50: The cells were removed from the flask and transferred to a 15 ml falcon tube and centrifuged at 500×g for 5 minutes. The medium on the cells that precipitated on the bottom of the falcon tube was discarded and 1 ml of new medium was added thereon and dissolved by means of a pipette. Then, the cells were seeded in 96-well plates to be monolayer within 24±2 hours and incubated at 37° C. in a 5% CO₂ incubator. When the cells were observed to be monolayer under an inverted microscope, the cells were processed. Vero cell line was used for Adenovirus and Poliovirus, Raw cell line was used for Murine norovirus. The substances whose non-toxic doses were determined were prepared with virus medium according to the volume to be used. 225 μl of virus medium was added to a new 96-well plate and 25 μl of virus was added to the first 6 wells of the 96-well plate and serial dilutions were made on a logarithmic scale (Log 10). The medium of the prepared monolayered cells was discarded. They were washed twice with virus medium. Serial dilutions prepared in a new 96-well plate were transferred on the cells and the medium volume was completed to 200 ul with the prepared non-toxic dose of the substance. Plates were incubated in a 37° C. 5% CO₂ incubator for 72 hours. At the end of the incubation period, the cytopathic effect (CPE) due to virus suspension was evaluated under an inverted microscope. The obtained results were evaluated by performing TCID50 calculation with Spearman-Karber method according to the following formulas.

$$M = Xk + d [ 0.5 - ( 1 / n ) ( r ) ] \qquad ( Formula 1 )$$

In Formula 1, Xk is the highest dilution dose, d is the difference between dilutions, n is the number of wells per dilution, ris the sum of (−) responses.

$$Mv = lg ( Va / Vc ) = lg ( Va ) - lg ( Vc ) \qquad ( Formula 2 )$$

In Formula 2, Mv is the antiviral activity value, 1 g (Va) is the logarithmic mean of two biological replicates for control tests, 1 g (Ve) is the logarithmic mean of two biological replicates for experimental groups

The antiviral efficacy of Lutetium Nitrate and Lutetium Chloride compounds against different types of viruses is shown in the table below.

| Substance Name | Adenovirus | Poliovirus | Murine | Bovine |
|---|---|---|---|---|
| and Concentration | Type 5 | Type 3 | Norovirus | Coronavirus |
| Lu(NO₃)₃ 750 ppm | 4.0 Log | 4.0 Log | 4.0 Log | 2.0 Log |
| Lu(NO₃)₃ 500 ppm | 4.0 Log | 4.0 Log | 4.0 Log | 2.0 Log |
| Lu(NO₃)₃ 250 ppm | 4.0 Log | 3.5 Log | 4.0 Log | 2.0 Log |
| LuCl₃ 750 ppm | 4.0 Log | 4.0 Log | 4.0 Log | 2.5 Log |
| LuCl₃ 500 ppm | 4.0 Log | 4.0 Log | 4.0 Log | 3.0 Log |
| LuCl₃ 250 ppm | 4.0 Log | 4.0 Log | 4.0 Log | 3.0 Log |

The results of studies carried out with LuBO₃, LuNO₃ and LuCl₃ demonstrate that antiviral efficacy is associated with the lutetium element. Therefore, it is evaluated that other lutetium salt compounds and/or products/formulations containing these salt compounds may have similar antiviral efficacy. In the analyses carried out for these three compounds, the efficacy concentration was detected to be in the range of 100 ppm to 1000 ppm. Therefore, it is evaluated that the antiviral activity value associated with Lutetium compound may also apply for other compounds/products/formulations containing the lutetium element.

Synthesis of lutetium borate:

The solution containing sodium hydroxide and boric acid (preferably in a 1:2 ratio) is preferably prepared in 50 mL of deionized water. Lutetium nitrate solution is prepared in polyvinyl alcohol with a total volume of preferably 50 mL. Lutetium nitrate solution is added into the prepared sodium hydroxide and boric acid solution at constant speed and stirred at 2000 rpm for the first 5 minutes and then at 1000 rpm for 25 minutes. The obtained lutetium borate solution can be used after being characterized.

Lutetium nitrate solution:

Lutetium nitrate is prepared preferably to be at an amount of 750 ppm, using deionized water.

Lutetium chloride solution:

Lutetium chloride is prepared preferably to be at an amount of 750 ppm, using deionized water.

It is possible to prepare and use the lutetium salt compounds studied within the scope of the invention and other lutetium salts at appropriate concentrations in different solvents by similar methods.

In the embodiment of the invention, solvents such as PVA, glucopan were used to better disperse the compounds to be formed during the preparation of lutetium borate. Within the scope of the invention, PVA and glucopan are particularly preferred. Because LuBO₃ does not show toxic effects on cells while suspending its salts.

### REFERENCES [1]. Ekmekyapar, Muhammed, and Şükrü Gürbüz. “Antiviral Drugs and Their Toxicities.” Eurasian Journal ofToxicology 1.3 (2018): 77-84.

[2]. Q .-L. Guan, Y .-H. Xing, J. Liu, W .-J. Wei, X. Wang, F .-Y. Bai, Application of multiple parallel perfused microbiorectors: synyhesis, characterization and cytotoxicity testing ofthe novel rare earth complexes with indole acid as a ligand, J. Inorg. Biochem. 128 (2013) 57-67.

[3]. S. Ban, S. Suzuki, K. Kubota, S. Ohshima, H. Satoh, H. Imada, Y. Ueda, Gastric mucosal status susceptible to lanthanum deposition in patients treated with dialysis and lanthanum carbonate, Ann. Diagn. Pathol. 26 (2017) 6-9.

What is claimed is:

1. A lutetium salt compound for providing an antiviral effect against DNA and RNA viruses on infected cells, wherein the DNA and RNA viruses comprise Adenovirus, Poliovirus, Herpes Simplex Type 1, Herpes Simplex Type 2. Coronavirus, and Norovirus.

2. The lutetium salt compound according to claim 1, wherein the lutetium salt compound is lutetium borate configured for providing the antiviral effect against the DNA and RNA viruses on infected suitable cell lines, and the infected suitable cell lines comprise HaCaT, Vero, and Raw 264.7.

3. The lutetium salt compound according to claim 1, wherein the lutetium salt compound is lutetium chloride.

4. The lutetium salt compound according to claim 1, wherein the lutetium salt compound is lutetium nitrate.

5. The lutetium salt compound according to claim 1, wherein the lutetium salt compound comprises a water-soluble or water-insoluble lutetium salt compound formed by an lutetium element with nitrate, sulfate, acetate, oxide, hydroxide, fluoride, carbonate, phosphate, or oxalate.

6. A product and/or a formulation produced for pharmaceutical, medical device, biocidal, and cosmetic purposes for providing the antiviral effect against the DNA and RNA viruses, comprising the lutetium salt compound according to claim 1.

7. The product and/or the formulation according to claim 6, wherein the product and/or the formulation a form of cream, spray, lotion, gel, capsule, tablet, toothpaste, mouthwash, mask, disinfectant, antiseptic, powder, polish, brightener, jelly, or foam pomade.

8. The product and/or the formulation according to claim 6, wherein the lutetium salt compound is lutetium borate configured for providing the antiviral effect against the DNA and RNA viruses on infected suitable cell lines, and the infected suitable cell lines comprise HaCaT, Vero, and Raw 264.7.

9. The product and/or the formulation according to claim 6, wherein the lutetium salt compound is lutetium chloride.

10. The product and/or the formulation according to claim 6, wherein the lutetium salt compound is lutetium nitrate.

11. The product and/or the formulation according to claim 6, wherein the lutetium salt compound comprises a water-soluble or water-insoluble lutetium salt compound formed by an lutetium element with nitrate, sulfate, acetate, oxide, hydroxide, fluoride, carbonate, phosphate, or oxalate.
